(19) United States
(12) Patent Application Publication
(10) Pub. No.: US 2024/0180992 A1
(43) Pub. Date: Jun. 6, 2024

(54) Control of tight junction through the use of polyphenols

(71) Applicant: Guilin Layn Natural Ingredients Corp., Guilin (CN)

(72) Inventors: Juan A. Javierre, Barcelona (ES); Maria Jose Fernandez Llibre, Rochester, MI (US)

(21) Appl. No.: 18/440,688

(22) Filed: Feb. 13, 2024

Related U.S. Application Data
(63) Continuation-in-part of application No. 17/094,318, filed on Nov. 10, 2020.
(60) Provisional application No. 63/089,827, filed on Oct. 9, 2020.

(51) Int. Cl. A61K 36/82; A23K 10/30; A23K 20/121; A23K 50/75; A61K 9/00; A61K 31/353; A61K 31/7048; A61K 36/16; A61P 3/02

(52) U.S. Cl. CPC A61K 36/82; A23K 10/30; A23K 20/121; A23K 50/75; A61K 9/0056; A61K 31/353; A61K 31/7048; A61K 36/16; A61P 3/02

(57) ABSTRACT

A method of controlling a tight junction between epithelial cells of an animal comprises providing the animal with a feed containing polyphenols in an amount effective to increase transmembrane protein levels between the epithelial cells thereby minimizing passage of macromolecules between the epithelial cells of the animal.

### CROSS-REFERENCE TO RELATED APPLICATION

The present application is a continuation-in-part of U.S. patent application Ser. No. 17/094,318, filed Nov. 10, 2020, which is based on and claims the benefit of U.S. provisional patent application Ser. No. 63/089,827, filed Oct. 9, 2020, the content of which are hereby incorporated by reference in their entirety.

### BACKGROUND

This disclosure relates to the use of polyphenols to control the tight junction between and among epithelial cells thereby controlling the passage of harmful macromolecules of microbial origin and/or antigens from the intestinal lumen towards the lamina propria of the epithelium in animals.

Polyphenols are plant-based compounds that are naturally found in fruits, vegetables, herbs, spices and other food sources such as tea, chocolate and wine. Polyphenols act as antioxidants, neutralizing harmful free radicals, and are also thought to reduce inflammation.

It is generally believed that polyphenols, although there are several thousand types of polyphenols, can be categorized in four main groups, flavonoids, phenolic acids, polyphenolic amides, and other polyphenols.

### SUMMARY

This disclosure relates to a method of controlling a tight junction between epithelial cells of an animal. The method comprises providing the animal with a feed containing polyphenols at a dose of about 25-100 ppm of feed to increase transmembrane protein levels between the epithelial cells thereby minimizing passage of macromolecules between the epithelial cells of the animal.

In one or more embodiments, a source of the polyphenols are botanical extracts comprising green tea extract and Glycine max extract.

In one or more embodiments, the polyphenols comprise epigallocatechin gallate, catechin, epigallocatechin, and genistein.

In one or more embodiments, a source of the polyphenols are botanical extracts comprising Camellia sinensis extract and Ginkgo biloba extract.

In one or more embodiments, the polyphenols comprise epigallocatechin gallate and rutin.

In one or more embodiments, the animal provided the feed has a monogastric intestinal system.

In one or more embodiments, the macromolecules are of microbial origin.

In one or more embodiments, the feed contains a carrier comprising soybean concentrate and limestone.

In one or more embodiments, the method increases a growth rate of the animal.

Another aspect of this disclosure relates to a method of increasing nutrient absorption in an animal. The method comprises providing the animal with a blend of polyphenols in an amount effective to increase transmembrane protein levels between the epithelial cells thereby minimizing passage of macromolecules between the epithelial cells of the animal.

In one or more embodiments, a source of the polyphenols are botanical extracts comprising green tea extract and Glycine max extract.

In one or more embodiments, the polyphenols comprise epigallocatechin gallate, catechin, epigallocatechin, and genistein.

In one or more embodiments, a source of the polyphenols are botanical extracts comprising Camellia sinensis extract and Ginkgo biloba extract.

In one or more embodiments, a concentration of Camellia sinensis extract is about 3.6% of the blend and a concentration of Ginkgo biloba extract is about 24% of the blend.

In one or more embodiments, the polyphenols comprise epigallocatechin gallate and rutin.

In one or more embodiments, a concentration of epigallocatechin gallate is about 1.62% of the blend and a concentration of rutin is about 6% of the blend.

In one or more embodiments, the blend is applied to a feed.

In one or more embodiments, the animal provided the blend has a monogastric intestinal system.

In one or more embodiments, the feed contains a carrier comprising soybean concentrate and limestone.

In one or more embodiments, the method increases a growth rate of the animal.

### DETAILED DESCRIPTION

This disclosure relates to the use of polyphenols to control the tight junction between epithelial cells thereby controlling the passage of harmful macromolecules of microbial origin or antigens of microbial origin from intestinal lumen towards the lamina propria.

For purposes of this disclosure, tight junctions are areas where the membranes of adjacent epithelial cells join together to form a barrier. The cell membranes are connected by strands of transmembrane proteins such as claudins and occludins. The tight junctions form a continuous intercellular barrier within the epithelium controlling the passage of substances from the intestinal lumen towards the lamina propria of the epithelium thereby selectively regulating movement across the epithelium.

The epithelial cells are responsible for taking up nutrients from the gut. Along with this responsibility in taking up nutrients, unwanted substances such as harmful macromolecules of microbial origin need to be controlled or blocked from entering the body through the epithelium. The more that such harmful macromolecules pass through the tight junction, the more the immune system of the animal needs to respond to the numbers of such harmful macromolecules entering the body. This response by the animal then takes up energy which otherwise could be used for positive effects on the animal's welfare which in turn will have an effect on commercial aspects for which the animal is being raised. Such commercial aspects include better growth, muscle mass gain, weight gain, egg production in the case of poultry, and meat quality to name a few. For purposes of this application, the phrase “harmful macromolecules” shall mean such macromolecules that have the herein described detrimental effect on the animal.

The method of this disclosure prevents/minimizes the entry of harmful macromolecules of microbial origin into the animal's body. Thus, the effect of such macromolecules is avoided. This results in the animal's immune system not having to expend energy to fight off the effect of such harmful macromolecules and reduces inflammatory responses. For example, this can lead to down regulation of MAPK and NF-κB pathways in the enterocyte and increased nutrient absorption by the epithelial cells.

The use of certain polyphenols as described further herein controls the pathways through which macromolecules enter the body. Such pathways are referred to as tight junctions which are areas between and among epithelial cells. A network of proteins extend between the epithelial cells. The proteins being affected by the method of this invention primarily include Claudin 1, Claudin 5, Occludin, and Zonula Occludens. Chelakkot et al. Experimental & Molecular Medicine (2018) 50:103. The polyphenols of this disclosure increase the abundance, the mass, of such proteins thus forming a tighter barrier type thereby preventing harmful macromolecules from passing through the epithelium.

This disclosure also relates to a method of making a feed supplement and feeding the polyphenol containing supplement to animals. By feeding animals the feed supplement of this disclosure the health and nutritional wellbeing of the animals is either maintained or increased positively by decreasing or eliminating the macromolecules from microbial origin from affecting the animal. The selected polyphenols are plant based and are selected to effectively increase the protein level in the animal's tight junctions of the epithelium layer.

The method of this disclosure can also be used as a treatment for leaky gut syndrome. By the phrase, leaky gut syndrome, as used herein, it is meant the condition in which the small intestine lining becomes damaged, allowing the macromolecules to pass between and among epithelial cells, resulting in cell damage or inflammation of the intestine. Once the macromolecules pass between cells, the animal's immune system recognizes them and triggers an immune response to destroy and remove the harmful macromolecules. This inflammatory process and immune system activation may consume significant amounts of energy and nutrients. While an appropriate inflammatory response is necessary, excessive or prolonged inflammation can become detrimental to the animal. Animals that suffer from chronic, prolonged inflammation may be unable to mount a successful immune response in the future. Chronic inflammation pulls nutrients and energy away from other key functions within the animal such as growth, reproduction, and meat or egg production.

The preferred animals for the polyphenol containing feed have a monogastric intestinal system and include without limitation poultry such as chickens, swine, horses, pets such as dogs and cats, and fish. Since the polyphenols of this disclosure are plant based such feed or feed supplements can be considered and promoted as natural and therefore healthy supplements for animals.

In one or more embodiments, the feed of this disclosure comprises a blend of polyphenol-rich botanical extracts, including but not limited to Vitis vinifera extract, Glycine max extract, Hibiscus sabdariffa extract, Camellia sinensis extract, green tea extract, Ginkgo biloba extract, Coffea arabica extract, Punica granatum extract, Vaccinium myrtillus, or Sophora japonica extract. The blend of polyphenol extracts may be any of the aforesaid alone or in combination.

The polyphenols obtained from said extracts include, but are not limited to quercetin, kaempferol, genistin, genistein, myricetin, daidzein, chlorogenic acid, caffeic acid, rutin, delphidin, cyaniding, epigallocatechin, epigallocatechin gallate, catechin, epicatechin, gallic acid, ellagic acid, punicalagins, procyanidin B or combinations thereof.

A blend may have a polyphenol and/or polyphenol extract concentration of less than 25%, 5%, or even 1% of blend. A feed may have a polyphenol and/or polyphenol extract concentration of less than 1% of feed. In one or more embodiments, the dose of polyphenols and/or polyphenol extracts may be 25-100 ppm of feed. For example, 50 ppm or 0.05 g/kg of feed.

In one or more embodiments, the feed comprises a blend of Camellia sinensis extract and Ginkgo biloba extract. For example, Camellia sinensis extract at a concentration of about 3.6% of blend and Ginkgo biloba extract at a concentration of about 24% of blend. In one or more embodiments, the polyphenols applied to the feed comprise epigallocatechin gallate and rutin. For example, epigallocatechin gallate at a concentration of about 1.62% of blend and rutin at a concentration of about 6% of blend.

In one or more embodiments, the feed comprises a blend of green tea extract and Glycine max extract. The polyphenols may comprise epigallocatechin gallate (EGCG), catechin, epigallocatechin, and genistein. For example, the dose of polyphenols may be 50 ppm or 0.05 g/kg of feed.

In one or more embodiments, the feed further comprises a carrier. The carrier may be used as a diluent to facilitate the distribution of polyphenols in the feed and is generally not an active ingredient. For example, a carrier is a mixture of soybean concentrate and limestone.

The blend of polyphenols is added continuously to the diet of the intended target animal; alternatively, they can also be administered mixed with drinking water.

### Example

In a trial involving about 2,000 broilers, a blend of green tea extract and Glycine max extract was added to the broiler feed and fed ad libitum for 6 weeks. The active ingredients (polyphenols) included epigallocatechin gallate (EGCG), catechin, epigallocatechin, and genistein. The polyphenols were provided at a dose of 50 ppm of feed. Results from trials using the polyphenol blend on broiler chickens are described in the tables below.

Table 1 below shows the relative abundance of Tight Junction proteins comparing a control group with a group of broilers fed the polyphenol feed of this disclosure.

TABLE 1
Relative abundance of Tight Junction proteins. Percent increase
of polyphenol-treated birds over the untreated controls

| | | | | Zonula |
|---|---|---|---|---|
| | Claudin1 | Claudin5 | Occludin | Occludens |
| Control | 100 | 100 | 100 | 100 |
| PF | 359 | 188 | 237 | 160 |

Table 2 below shows the comparative weight gain of broilers fed the polyphenols of this disclosure in comparison to a control group of broilers. The numerical values in Table 2 are mean values for both the control and the polyphenol fed group of broilers. Measurements were done weekly from week 1 to week 6. 0 is day one of the first week, the day the trial started.

TABLE 2
Body weight of broilers in grams. Feed provided ad libitum

| Week | 0 | 1 | 2 | 3 | 4 | 5 | 6 |
|---|---|---|---|---|---|---|---|
| Control | 43.8 | 154 | 359 | 720 | 1237 | 1797 | 2361 |
| Group | | | | | | | |
| Polyphenol | 43.2 | 161 | 379 | 755 | 1322 | 1902 | 2569 |
| Feed | | | | | | | |
| Group | | | | | | | |

Table 3 below shows feed conversion ratio (FCR), which is a measure of efficiency. (Feed eaten (kg)/body mass gain (kg))

TABLE 3
Cumulative FCR on a weekly basis

| Week | 1 | 2 | 3 | 4 | 5 | 6 |
|---|---|---|---|---|---|---|
| Control | 0.819 | 1.301 | 1.401 | 1.493 | 1.640 | 1.732 |
| Group | | | | | | |
| Polyphenol | 0.786 | 1.265 | 1.351 | 1.437 | 1.576 | 1.707 |
| Feed | | | | | | |
| Group | | | | | | |

1. A method of controlling a tight junction between epithelial cells of an animal, the method comprising:
providing the animal with a feed containing polyphenols at a dose of about 25-100 ppm of feed to increase transmembrane protein levels between the epithelial cells thereby minimizing passage of macromolecules between the epithelial cells of the animal.

2. The method of claim 1 wherein a source of the polyphenols are botanical extracts comprising green tea extract and Glycine max extract.

3. The method of claim 1 wherein the polyphenols comprise epigallocatechin gallate, catechin, epigallocatechin, and genistein.

4. The method of claim 1 wherein a source of the polyphenols are botanical extracts comprising Camellia sinensis extract and Ginkgo biloba extract.

5. The method of claim 1 wherein the polyphenols comprise epigallocatechin gallate and rutin.

6. The method of claim 1 wherein the animal provided the feed has a monogastric intestinal system.

7. The method of claim 1 wherein the macromolecules are of microbial origin.

8. The method of claim 1 wherein the feed further contains a carrier comprising soybean concentrate and limestone.

9. The method of claim 1 wherein the method increases a growth rate of the animal.

10. A method of increasing nutrient absorption in an animal, the method comprising:
providing the animal with a blend of polyphenols in an amount effective to increase transmembrane protein levels between the epithelial cells thereby minimizing passage of macromolecules between the epithelial cells of the animal.

11. The method of claim 10 wherein a source of the polyphenols are botanical extracts comprising green tea extract and Glycine max extract.

12. The method of claim 11 wherein the polyphenols comprise epigallocatechin gallate, catechin, epigallocatechin, and genistein.

13. The method of claim 10 wherein a source of the polyphenols are botanical extracts comprising Camellia sinensis extract and Ginkgo biloba extract.

14. The method of claim 13 wherein a concentration of Camellia sinensis extract is about 3.6% of the blend and a concentration of Ginkgo biloba extract is about 24% of the blend.

15. The method of claim 13 wherein the polyphenols comprise epigallocatechin gallate and rutin.

16. The method of claim 15 wherein a concentration of epigallocatechin gallate is about 1.62% of the blend and a concentration of rutin is about 6% of the blend.

17. The method of claim 10 wherein the blend is applied to a feed.

18. The method of claim 10 wherein the animal provided the blend has a monogastric intestinal system.

19. The method of claim 17 wherein the feed further contains a carrier comprising soybean concentrate and limestone.

20. The method of claim 10 wherein the method increases a growth rate of the animal.
